(19) United States
(12) Patent Application Publication
(10) Pub. No.: US 2019/0091359 A1
(43) Pub. Date: Mar. 28, 2019

(54) VACUUM STERILIZATION DEVICE

(71) Applicant: Min-Teh Shih, New Taipei City 24259 (TW)

(72) Inventor: Min-Teh Shih, New Taipei City 24259 (TW)

(21) Appl. No.: 15/713,992

(22) Filed: Sep. 25, 2017

(51) Int. Cl. A61L 2/20

(52) U.S. Cl. CPC A61L 2/202; A61L 2209/212; A61L 2202/23

(57) ABSTRACT

A vacuum sterilization device includes a vacuum pump, an ozone generator, a connector and a panel. The vacuum pump is connected to a vacuum electro-magnetic valve, and the vacuum electro-magnetic valve is connected to the connector. The ozone generator is connected to an ozone electro-magnetic valve which is connected to the connector as well. The vacuum sterilization device vacuums a pack and then introduces ozone into the pack. After the ozone sterilizes the interior of the pack, the vacuum pump sucks the ozone from the pack. The users can set the time required and expected results of the ozone on the panel.

### BACKGROUND OF THE INVENTION

### 1. Fields of the Invention

The present invention relates to a vacuum sterilization device, and more particularly, to a vacuum sterilization device that vacuums the pack and introduces ozone to sterilize the interior of the pack.

### 2. Descriptions of Related Art

Ozone is used for sterilization to kill germs, bacteria and fungus, and to degrade toxic gas. The ozone is perfect to remove smoke smell, alcohol smell, and stench from air. Besides, the ozone with a certain level of density is bale to enter into human cells and damage the protein enzyme in the cells. When ozone enters into the nuclei, it can damage DNA in the nuclei to kill the microorganism or to stop reproduction of the microorganism. Therefore, ozone is widely used to sterilize packs before goods are packed in the packs. However, there is no way to retrieve ozone after the ozone is used for sterilization.

The present invention intends to provide a vacuum sterilization device that vacuums and retrieves the ozone after sterilization.

### SUMMARY OF THE INVENTION

The present invention relates to a vacuum sterilization device which comprises a vacuum pump, and an initialization pipe is connected between the vacuum pump and a vacuum electro-magnetic valve. The vacuum electro-magnetic valve has a pressure-detection pipe. An ozone generator is connected with an ozone electro-magnetic valve and generates ozone. An air pump pumps the ozone out from the ozone generator. A connector is a three-way connector and connected to the vacuum electro-magnetic valve and the ozone electro-magnetic valve.

The vacuum pump vacuums a pack and the pressure-detection pipe creates a signal to stop operation of the vacuum pump and the vacuum electro-magnetic valve after the pack is vacuumed. And then, the air pump, the ozone generator and the ozone electro-magnetic valve are activated to introduce ozone into the pack to sterilize the interior of the pack. The ozone in the pack is retrieved by the vacuum pump and transferred into oxygen via the initialization pipe. The pressure-detection pipe sends a signal to the panel to acknowledge the users when vacuuming and retrieval of ozone are completed. The time required and the expected results of the ozone can be set on the panel by the users.

The primary object of the present invention is to provide a vacuum sterilization device that vacuums and sterilize by ozone a pack so that the pack is ready for storage of goods or food.

Another object of the present invention is to provide a vacuum sterilization device wherein the ozone can be transferred into oxygen after being used. When the sterilization is completed, the users are acknowledged by the device. The time required and the expected results of the ozone can be set by the users.

The present invention will become more apparent from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the vacuum sterilization device of the present invention;

FIG. 2 is a perspective view to show the vacuum sterilization device of the present invention, wherein the case is removed;

FIG. 3 is a perspective view to show the vacuum sterilization device of the present invention, wherein a pack is connected to the hose, and

FIG. 4 is a perspective view to show the vacuum sterilization device of the present invention, wherein ozone is introduced into the pack.

### DETAILED DESCRIPTION OF THE PREFERRED EMBODIMENT

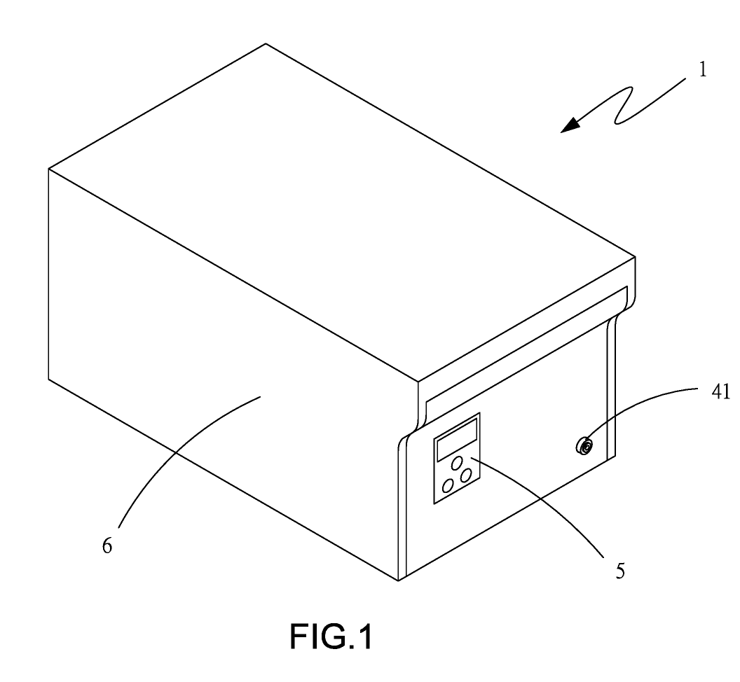

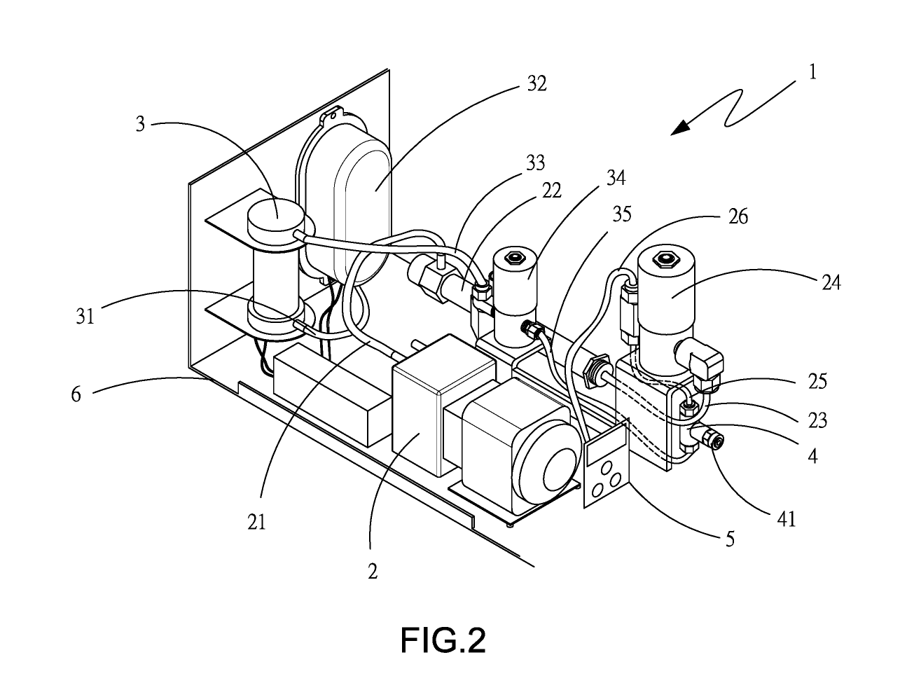

Referring to FIGS. 1 and 2, the vacuum sterilization device 1 of the present invention comprises a vacuum pump 2, an ozone generator 3, a connector 4 and a panel 5. The vacuum pump 2 and the ozone generator 3 are received in a case 6. The connector 4 and the panel 5 are connected to the case 6.

The vacuum pump 2 is connected to a suction pipe 21, and the suction pipe 21 is connected with an initialization pipe 22 which heats ozone and transfers the ozone into oxygen. The initialization pipe 22 is connected to a transfer pipe 23 which is connected to a vacuum electro-magnetic valve 24. The vacuum electro-magnetic valve 24 has a connection pipe 25 and a pressure-detection pipe 26. The connection pipe 25 is connected to the connector 4 which is a three-way connector. In this embodiment, the connection pipe 25 is connected to the top position of the connector 4. The vacuum electro-magnetic valve 24 is connected to the panel 5 by a pressure-detection pipe 26. The ozone generator 3 has a pumping pipe 31 which is connected to an air pump 32. The ozone generator 3 includes an ozone pipe 33 which is connected with an ozone electro-magnetic valve 34. The ozone electro-magnetic valve 34 is connected to the bottom position of the connector 4 by an outlet pipe 35. The connector 4 includes a connection end 41.

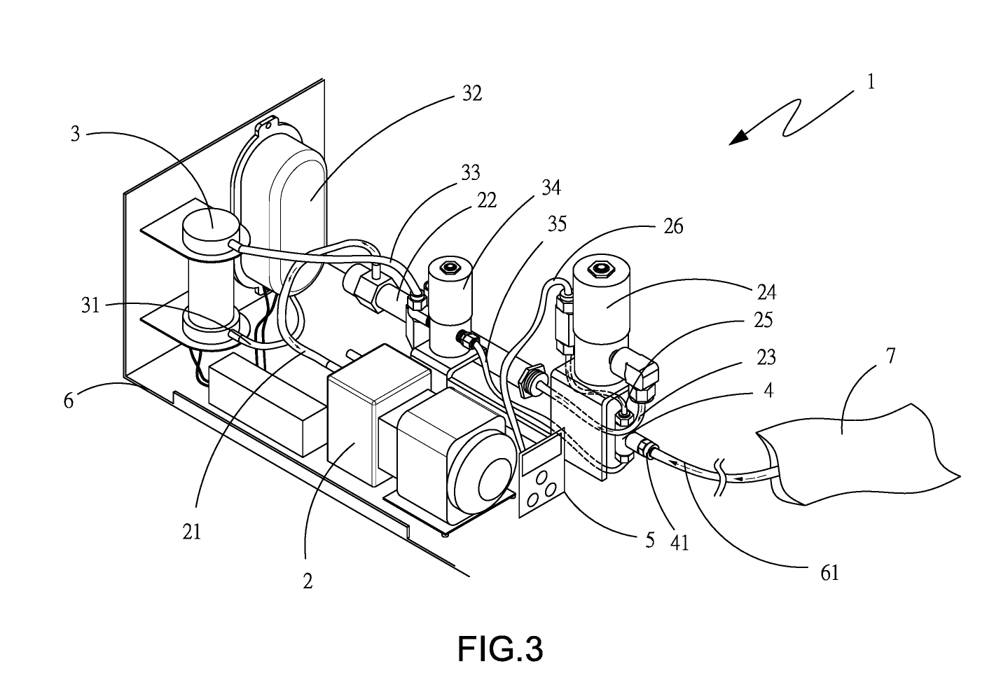

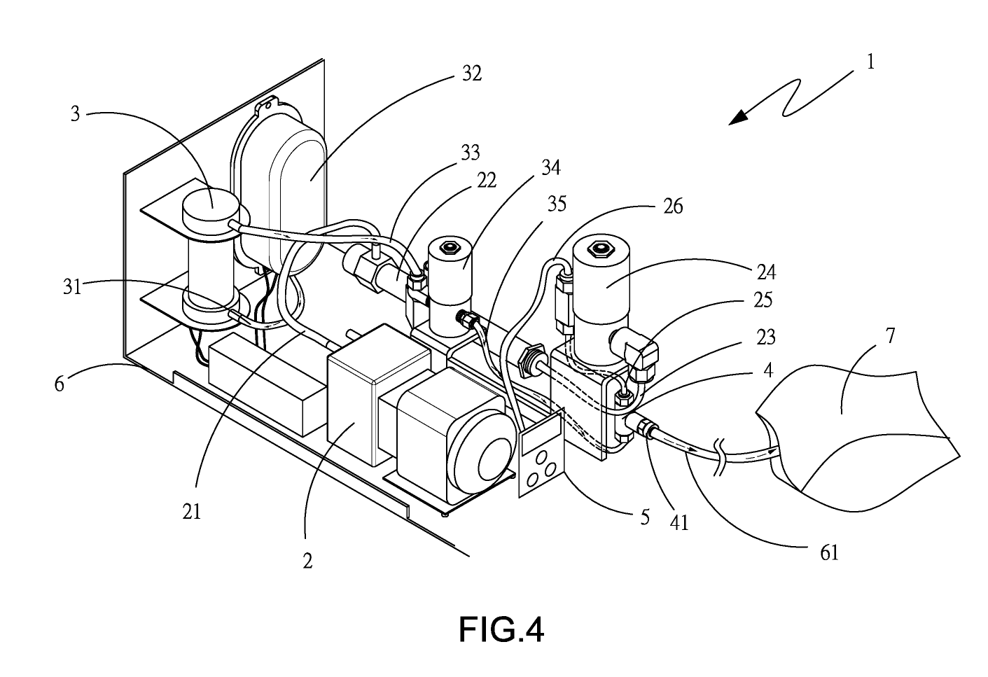

As shown in FIGS. 3 and 4, the vacuum sterilization device 1 is connected to a hose 61, wherein the first end of the hose 61 is connected to the connection end 41, and the second end of the hose 61 is located in the pack 7. The vacuum pump 2 is activated to vacuum the interior of the pack 7, the air in the pack 7 is sent to the connection end 41 of the connector 4 via the hose 61. The air in the connector 4 is then sent to the vacuum electro-magnetic valve 24 via the connection pipe 25. The air passes through the vacuum electro-magnetic valve 24 and is sent to the initialization pipe 22 and the suction pipe 21 via the transfer pipe 23, and then sucked by the vacuum pump 2. The pressure-detection pipe 26 sends a signal to the panel 5 and the vacuum electro-magnetic valve 24 when the pack 7 is vacuumed. The vacuum electro-magnetic valve 24 is automatically shut off. The panel 5 simultaneously shots off the vacuum pump 2. The ozone generator 3, the air pump 32 and the ozone electro-magnetic valve 34 are activated. The air pump 32 generates pressured air which is sent to the ozone generator 3 by the pumping pipe 31. The ozone of the ozone generator 3 is sent to the ozone electro-magnetic valve 34 by the ozone pipe 33. The ozone passes through the ozone electro-magnetic valve 34 and sent to the connection end 41 of the connector 4 by the outlet pipe 35. The ozone is then sent into the pack 7 to proceed sterilization processes via the hose 61. When the pack 7 is filled with ozone, the pressure-detection pipe 26 sends a signal to the panel 5 which creates a signal to acknowledge the users that the pack 7 is filled with ozone. The users can set the time required for the operation time of the ozone so that the users know when the operation of the ozone is completed from the panel 5. As shown in FIG. 3, after the pack 7 is processed by the ozone completely, the vacuum pump 2 is activated again to suck the ozone out from the pack 7 and sets the pack 7 to be vacuum status.

The ozone sucked from the pack 7 is sent to the connection end 41 of the connector 4 via the hose 61. The ozone in the connector 4 is then sent to the vacuum electro-magnetic valve 24 via the connection pipe 25. The ozone passes through the vacuum electro-magnetic valve 24 and is sent to the initialization pipe 22 via the transfer pipe 23. The initialization pipe 22 heats and transfers the ozone into oxygen which is sucked by the vacuum pump 2 via the suction pipe 21. Therefore, the used ozone is transferred into oxygen after being used. Therefore, vacuum sterilization device 1 is able to fill ozone into pack 7 to sterilize the pack 7 by the ozone generator 3. When the sterilization is completed, the vacuum pump 2 sucks the ozone out from the pack 7 and vacuums the pack 7 so that the pack 7 is ready to store goods or food. By using the pressure-detection pipe 26, the vacuum electro-magnetic valve 24 and the ozone electro-magnetic valve 34, the vacuum electro-magnetic valve 24 is automatically shut off, and the information is acknowledged by the users from the panel 5.

While we have shown and described the embodiment in accordance with the present invention, it should be clear to those skilled in the art that further embodiments may be made without departing from the scope of the present invention.

What is claimed is:

1. A vacuum sterilization device comprising:
a vacuum pump;
an initialization pipe connected between the vacuum pump and a vacuum electro-magnetic valve, the vacuum electro-magnetic valve having a pressure-detection pipe;
an ozone generator connected with an ozone electro-magnetic valve, the ozone generator generating ozone and an air pump pumping the ozone out from the ozone generator;
a connector being a three-way connector, the connector connected to the vacuum electro-magnetic valve and the ozone electro-magnetic valve, wherein the vacuum pump vacuums a pack and the pressure-detection pipe creates a signal to stop operation of the vacuum pump and the vacuum electro-magnetic valve after the pack is vacuumed, and then the air pump, the ozone generator and the ozone electro-magnetic valve are activated to introduce ozone into the pack to sterilize interior of the pack, the ozone in the pack is retrieved by the vacuum pump, the pressure-detection pipe sends a signal to users when vacuuming and retrieval of ozone are completed.

2. The vacuum sterilization device as claimed in claim 1, wherein the vacuum pump and the ozone generator are received in a case, the connector and a panel are connected to the case.

3. The vacuum sterilization device as claimed in claim 1, wherein the vacuum pump is connected to the initialization pipe by a suction pipe, the initialization pipe is connected to the vacuum electro-magnetic valve by a transfer pipe, the vacuum electro-magnetic valve is connected to the connector by a connection pipe.

4. The vacuum sterilization device as claimed in claim 1, wherein the air pump is connected to the ozone generator by a pumping pipe, the ozone generator is connected to the ozone electro-magnetic valve by an ozone pipe, the ozone electro-magnetic valve is connected to the connector by an outlet pipe.

5. The vacuum sterilization device as claimed in claim 2, wherein the panel includes a display signal to show that the vacuuming and retrieval of ozone are completed.

6. The vacuum sterilization device as claimed in claim 5, wherein the vacuum electro-magnetic valve is connected to the panel by the pressure-detection pipe.

7. The vacuum sterilization device as claimed in claim 1, wherein the connector includes a connection end.

8. The vacuum sterilization device as claimed in claim 7, wherein the connection end is connected to a first end of a hose, a second end of the hose is located in the pack.
